(19) United States
(12) Patent Application Publication
(10) Pub. No.: US 2006/0292100 A1
(43) Pub. Date: Dec. 28, 2006

(54) Aqueous phospholipid-containing carrier systems for water-insoluble materials

(75) Inventors: Nghi Van Nguyen, Edison, NJ (US); David W. Cannell, Plainfield, NJ (US); Cynthia Chong Espino, Princeton, NJ (US); Sawa Hashimoto, Westfield, NJ (US); Katherine Natalie Barger, Cranford, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'OREAL, Paris (FR)

(21) Appl. No.: 11/154,437

(22) Filed: Jun. 16, 2005

(51) Int. Cl. A61K 8/55

(52) U.S. Cl. 424/70.12; 424/70.23

(57) ABSTRACT

The present invention is drawn to a carrier composition containing: (a) at least one phospholipid; (b) at least one nonionic surfactant; (c) at least one anionic silicone; and (d) at least one water-insoluble material, and wherein the composition, when combined with an aqueous phase, forms an aqueous delivery system which is both stable, and clear to slightly hazy/limpid in appearance.

### BACKGROUND OF THE INVENTION

The present invention relates to a novel carrier system based on a combination of at least one phospholipid, at least one nonionic surfactant, and at least one anionic silicone, wherein the carrier system allows water-insoluble materials to be incorporated into aqueous solutions.

Certain water-insoluble ingredients which are oftentimes desirable for the treatment of keratinous substrates are inherently difficult to incorporate into aqueous systems such as shampoos and conditioners without forming a traditional emulsion in either cream or lotion form. Moreover, many of these water-insoluble ingredients suppress lathering which makes the use of aqueous systems such as shampoos and body washes less desirable to consumers. Even in those aqueous systems which do employ these types of water-insoluble ingredients, their presence is minimal due to various performance drawbacks such as poor spreadability, foaming, removal and rinsing or, in the case of styling products, difficulties in removal via shampooing.

Also, when formulating clear to slightly limpid aqueous delivery systems for use in treating keratinous substrates, water-insoluble compounds do not lend themselves to being used therein, due to their inability to significantly associate with the water present in the system.

Thus, there remains a need for an aqueous delivery system which can carry water-insoluble materials while remaining both stable and clear, to slightly limpid, in appearance.

### SUMMARY OF THE INVENTION

In order to achieve these and other advantages, the present invention is drawn to a carrier composition containing:
(a) at least one phospholipid;
(b) at least one nonionic surfactant;
(c) at least one anionic silicone; and
(d) at least one water-insoluble material,
   wherein the composition, when combined with an aqueous phase, forms an aqueous delivery system which is both stable, and clear to slightly hazy/limpid in appearance.

In another embodiment, the present invention is also drawn to a process for making an aqueous delivery system which is both stable, and clear to slightly limpid in appearance, involving the steps of:
(a) providing a carrier composition containing: (i) at least one phospholipid, (ii) at least one nonionic surfactant and, (iii) at least one anionic silicone;
(b) providing at least one water-insoluble ingredient;
(c) optionally, heating the composition of step (a) to form a heated mixture;
(d) adding (b) to either step (a), step (c) or both step (a) and (c);
(e) adding an aqueous solution to either (c) or (d) to form a diluted mixture; and
(f) cooling the diluted mixture to form the aqueous delivery system.

Finally, in yet another embodiment, the present invention is drawn to a process for treating a keratinous substrate by contacting the substrate with an aqueous delivery system containing the above-disclosed carrier composition.

### DETAILED DESCRIPTION

Other than in the operating examples, or where otherwise indicated, all numbers expressing quantities of ingredients and/or reaction conditions are to be understood as being modified in all instances by the term “about”.

The term “water-insoluble” means those compounds which are either completely or partially insoluble in water.

The term “carried” means that the aqueous delivery system containing the water-insoluble ingredients is both stable and clear, to slightly limpid, in appearance.

“Keratinous substrate” as defined herein may be human keratinous fiber, and may be chosen from, for example, hair, eyelashes, and eyebrows, as well as the stratum corneum of the skin and nails.

Advantageously, the present invention allows water-insoluble materials or ingredients to be carried in an aqueous solution. No alcohol is required to render the system stable and clear, to slightly limpid, in appearance.

The carrier composition of the invention is easy to formulate and gentle on the hair, skin, or eyelashes because the surfactants used therein are generally mild.

The compositions and delivery systems of the present invention readily deliver water-insoluble ingredients to the targeted keratinous substrate. Accordingly, these compositions and delivery systems can be used in hair shampoos, conditioners, hair dyeing compositions, including oxidative dyes and bleaches, permanent waving compositions, curl relaxing compositions, hair setting compositions, bath and body products, sunscreens, cosmetics, skin moisturizers, and the like.

These systems can also be used to deliver active water-insoluble pharmaceutical ingredients, particularly in topical applications. Such systems could further help protect against oxidation and rancidity by protecting sensitive ingredients in pharmaceuticals or foods.

Without being bound to a particular theory, the inventors believe that the aqueous delivery system is in the form of a microemulsion whereby the anionic silicone forms an ion pair or pseudo-soap with the phospholipids which is then coupled to the aqueous solution as a mixed micelle (or other organized structure) by the nonionic surfactant. This structure is believed to be of sufficient stability and size to provide hydrophilic regions which carry the water-insoluble ingredients. These microemulsions range from pourable liquids to firm ringing gels.

The present invention provides for the use of conventional organic phospholipids. Particularly preferred organic phospholipids include lecithins. Lecithins are mixtures of phospholipids, i.e., of diglycerides of fatty acids linked to an ester of phosphoric acid. Preferably, lecithins are diglycerides of stearic, palmitic, and oleic acids linked to the choline ester of phosphoric acid. Lecithin is usually defined either as pure phosphatidyl cholines or as crude mixtures of phospholipids which include phosphatidyl choline, phosphatidyl serine, phosphatidyl ethanolamine, phosphatidyl inositol, other phospholipids, and a variety of other compounds such as fatty acids, triglycerides, sterols, carbohydrates, and glycolipids.

The lecithin used in the present invention may be present in the form of a liquid, powder, or granules. Lecithins useful in the invention include, but are not limited to, soy lecithin and hydroxylated lecithin. For example, ALCOLEC S is a fluid soy lecithin, ALCOLEC F 100 is a powder soy lecithin, and ALCOLEC Z3 is a hydroxylated lecithin, all of which are available from the American Lecithin Company.

Other than lecithins, additional examples of phospholipids which may be useful in the present invention include, but are not limited to, multifunctional biomimetic phospholipids. For example, the following multifunctional biomimetic phospholipids manufactured by Uniqema Industries may be useful: PHOSPHOLIPID PTC, PHOSPHOLIPID CDM, PHOSPHOLIPID SV, PHOSPHOLIPID GLA, and PHOSPHOLIPID EFA.

In the present invention, the organic phospholipid is preferably used in an amount of from greater than 0% to 30% by weight, preferably from greater than 0% to 10% by weight, and more preferably from greater than 0% to 5% by weight, based on the weight of the composition as a whole. Preferably, the carrier composition of the present invention, when combined with water, forms a clear solution, though the purpose of the invention is achieved just as effectively with a slightly cloudy/limpid solution.

In general, nonionic surfactants having a Hydrophilic-Lipophilic Balance (HLB) of from 8 to 20, are contemplated for use by the present invention. Nonlimiting examples of nonionic surfactants useful in the compositions of the present invention are disclosed in McCutcheon's “Detergents and Emulsifiers,” North American Edition (1986), published by Allured Publishing Corporation; and McCutcheon's “Functional Materials,” North American Edition (1992); both of which are incorporated by reference herein in their entirety.

Examples of nonionic surfactants useful herein include, but are not limited to, alkoxylated derivatives of the following: fatty alcohols, alkyl phenols, fatty acids, fatty acid esters and fatty acid amides, wherein the alkyl chain is in the C12-C50 range, preferably in the C16-C40 range, more preferably in the C24 to C40 range, and having from about 1 to about 110 alkoxy groups. The alkoxy groups are selected from the group consisting of C2-C6 oxides and their mixtures, with ethylene oxide, propylene oxide, and their mixtures being the preferred alkoxides. The alkyl chain may be linear, branched, saturated, or unsaturated. Of these alkoxylated non-ionic surfactants, the alkoxylated alcohols are preferred, and the ethoxylated alcohols and propoxylated alcohols are more preferred. The alkoxylated alcohols may be used alone or in mixtures thereof. The alkoxylated alcohols may also be used in mixtures with those alkoxylated materials disclosed herein-above.

Other representative examples of such ethoxylated fatty alcohols include laureth-3 (a lauryl ethoxylate having an average degree of ethoxylation of 3), laureth-23 (a lauryl ethoxylate having an average degree of ethoxylation of 23), ceteth-10 (a cetyl alcohol ethoxylate having an average degree of ethoxylation of 10) steareth-10 (a stearyl alcohol ethoxylate having an average degree of ethoxylation of 10), and steareth-2 (a stearyl alcohol ethoxylate having an average degree of ethoxylation of 2), steareth-100 (a stearyl alcohol ethoxylate having an average degree of ethoxylation of 100), beheneth-5 (a behenyl alcohol ethoxylate having an average degree of ethoxylation of 5), beheneth-10 (a behenyl alcohol ethoxylate having an average degree of ethoxylation of 10), and other derivatives and mixtures of the preceding.

Also available commercially are Brij® nonionic surfactants from ICI Specialty Chemicals, Wilmington, Del. Typically, Brij® is the condensation products of aliphatic alcohols with from about 1 to about 54 moles of ethylene oxide, the alkyl chain of the alcohol being typically a linear chain and having from about 8 to about 22 carbon atoms, for example, Brij 72 (i.e., Steareth-2) and Brij 76 (i.e., Steareth-10).

Also useful herein as nonionic surfactants are alkyl glycosides, which are the condensation products of long chain alcohols, e.g. C8-30 alcohols, with sugar or starch polymers. These compounds can be represented by the formula (S)n —O—R wherein S is a sugar moiety such as glucose, fructose, mannose, galactose, and the like; n is an integer of from about 1 to about 1000, and R is a C8-30 alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants are alkyl polyglucosides wherein S is a glucose moiety, R is a C8-20 alkyl group, and n is an integer of from about 1 to about 9. Commercially available examples of these surfactants include decyl polyglucoside (available as APG® 325 CS) and lauryl polyglucoside (available as APG® 600CS and 625 CS), all the above-identified polyglucosides APG® are available from Cognis, Ambler, Pa. Also useful herein are sucrose ester surfactants such as sucrose cocoate and sucrose laurate.

Other nonionic surfactants suitable for use in the present invention are glyceryl esters and polyglyceryl esters, including but not limited to, glyceryl monoesters, preferably glyceryl monoesters of C16-C22 saturated, unsaturated and branched chain fatty acids such as glyceryl oleate, glyceryl monostearate, glyceryl monoisostearate, glyceryl monopalmitate, glyceryl monobehenate, and mixtures thereof, and polyglyceryl esters of C16-C22 saturated, unsaturated and branched chain fatty acids, such as polyglyceryl-4 isostearate, polyglyceryl-3 oleate, polyglyceryl-2 sesquioleate, triglyceryl diisostearate, diglyceryl monooleate, tetraglyceryl monooleate, and mixtures thereof.

Also useful herein as nonionic surfactants are sorbitan esters. Preferable are sorbitan esters of C16-C22 saturated, unsaturated and branched chain fatty acids. Because of the manner in which they are typically manufactured, these sorbitan esters usually comprise mixtures of mono-, di-, tri-, etc. esters. Representative examples of suitable sorbitan esters include sorbitan monooleate (e.g., SPAN® 80), sorbitan sesquioleate (e.g., Arlacel® 83 from ICI Specialty Chemicals, Wilmington, Del.), sorbitan monoisostearate (e.g., CRILL® 6 from Croda, Inc., Parsippany, N.J.), sorbitan stearates (e.g., SPAN® 60), sorbitan trioleate (e.g., SPAN® 85), sorbitan tristearate (e.g., SPAN® 65), sorbitan dipalmitates (e.g., SPAN® 40), and sorbitan isostearate. Sorbitan monoisostearate and sorbitan sesquioleate are particularly preferred emulsifiers for use in the present invention.

Also suitable for use herein are alkoxylated derivatives of glyceryl esters, sorbitan esters, and alkyl polyglycosides, wherein the alkoxy groups is selected from the group consisting of C2-C6 oxides and their mixtures, with ethoxylated or propoxylated derivatives of these materials being the preferred. Nonlimiting examples of commercially available ethoxylated materials include TWEEN® (ethoxylated sorbitan mono-, di- and/or tri-esters of C12 to C18 fatty acids with an average degree of ethoxylation of from about 2 to about 20).

Preferred nonionic surfactants are those formed from a fatty alcohol, a fatty acid, or a glyceride with a C₄ to C₃₆ carbon chain, preferably a C₁₂ to C₁₈ carbon chain, more preferably a C₁₆ to C₁₈ carbon chain, derivatized to yield an HLB of at least 8. HLB is understood to mean the balance between the size and strength of the hydrophilic group and the size and strength of the lipophilic group of the surfactant. Such derivatives can be polymers such as ethoxylates, propoxylates, polyglucosides, polyglycerins, polylactates, polyglycolates, polysorbates, and others that would be apparent to one of ordinary skill in the art. Such derivatives may also be mixed polymers of the above, such as ethoxylate/propoxylate species, where the total HLB is preferably greater than or equal to 8. Preferably the nonionic surfactants contain ethoxylate in a molar content of from 10-25, more preferably from 10-20 moles.

The nonionic surfactant will typically be present in the composition in an amount of from greater than 0% to 70% by weight, preferably from greater than 0% to 40% by weight, and more preferably from greater than 0% to 20% by weight, based on the weight of the composition as a whole.

In general, non-limiting examples of anionic silicones which may be used in the present invention include silicone carboxylates, silicone phosphates, silicone sulfates, silicone sulfosuccinates, and silicone sulfonates.

Suitable silicone carboxylates may be chosen from water soluble silicone compounds comprising at least one carboxylic acid group, oil soluble silicone compounds comprising at least one carboxylic acid group, water-dispersible silicone compounds comprising at least one carboxylic acid group, and silicone compounds comprising at least one carboxylic acid group which are soluble in organic solvents. In one embodiment, the at least one silicone compound comprising at least one carboxylic acid group further comprises at least one alkoxylated chain, wherein the at least one alkoxy group may be chosen from terminal alkoxy groups, pendant alkoxy groups, and alkoxy groups which are intercalated in the skeleton of the at least one silicone compound. Non-limiting examples of at least one alkoxy group include ethylene oxide groups and propylene oxide groups.

The at least one carboxylic acid group may be chosen from terminal carboxylic acid groups and pendant carboxylic acid groups. Further, the at least one carboxylic acid may be chosen from carboxylic acid groups in free acid form, i.e., —COOH, and carboxylic acid groups in salt form, i.e., —COOM, wherein M may be chosen from inorganic cations, such as, for example, potassium cations and sodium cations, and organic cations.

In one embodiment, the at least one silicone compound comprising at least one carboxylic acid group is chosen from silicone compounds of formula (I) and salts thereof: 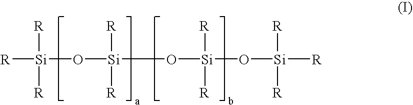
wherein: a is an integer ranging from 1 to 100; b is an integer ranging from 0 to 500; R, which may be identical or different, are each chosen from optionally substituted hydrocarbon groups comprising from 1 to 9 carbon atoms, optionally substituted phenyl groups, and groups of formula (II): 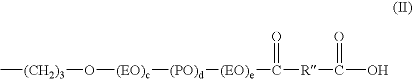
wherein: c, d, and e, which may be identical or different, are each integers ranging from 0 to 20; EO is an ethylene oxide group; PO is a propylene oxide group; and R″ is chosen from optionally substituted divalent hydrocarbons, such as alkylene groups and alkenylene groups comprising from 2 to 22 carbon atoms, and optionally substituted divalent aromatic groups, such as groups of formula (III): 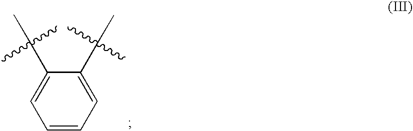
and groups of formula (IV): 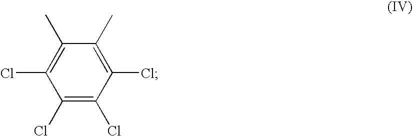
with the proviso that at least one of the R groups is chosen from groups of formula (II) and with the further proviso that when only one of the R groups is chosen from groups of formula (II), the other R groups are not all methyl groups.

Non-limiting examples of the at least one silicone compound include those commercially available from Noveon under the name Ultrasil® CA-1 Silicone and Ultrasil® CA-2 Silicone, both of which correspond to formula (V) below. This silicone carboxylate is sold in the free acid form as an emulsifier and dispersing aid for complexing fatty cationic amines and quaternary amines. Thus, in one embodiment, the at least one silicone compound is chosen from silicone compounds of formula (V) and salts thereof: 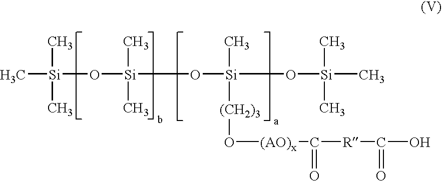
wherein: a is an integer ranging from 1 to 100; b is an integer ranging from 0 to 500; AO is chosen from groups of formula (VI):
-(EO)_{c}-(PO)_{d}-(EO)ₑ- (VI)
wherein: c, d, and e, which may be identical or different, are each integers ranging from 0 to 20; EO is an ethylene oxide group; and PO is a propylene oxide group; x is an integer ranging from 0 to 60; R″ is chosen from optionally substituted divalent hydrocarbons, such as alkylene groups and alkenylene groups comprising from 2 to 22 carbon atoms, and optionally substituted divalent aromatic groups, such as groups of formula (III): 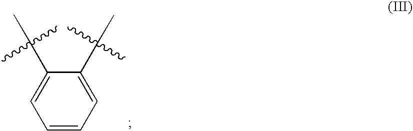
and groups of formula (IV):

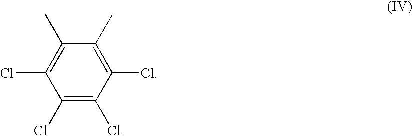

Non-limiting examples of the at least one silicone compound include those described in U.S. Pat. Nos. 5,248,783 and 5,739,371, the disclosures of which are incorporated herein by reference, and which are silicone compounds of formula (I).

Suitable silicone phosphates may be chosen from water-soluble silicone compounds comprising at least one phosphate group, oil soluble silicone compounds comprising at least one phosphate group, water-dispersible silicone compounds comprising at least one phosphate group, and silicone compounds comprising at least one phosphate group which are soluble in organic solvents.

In one embodiment, the at least one silicone compound comprising at least one phosphate group further comprises at least one alkoxylated chain, wherein the at least one alkoxy group may be chosen from terminal alkoxy groups, pendant alkoxy groups, and alkoxy groups which are intercalated in the skeleton of the at least one silicone compound. Non-limiting examples of at least one alkoxy group include ethylene oxide groups (“EO”═—CH₂—CH₂—O—) and propylene oxide groups (“PO”═C₃H₆O).

The at least one phosphate group may be chosen from terminal phosphate groups and pendant phosphate groups. Further, the at least one phosphate group may be chosen from groups of formula —O—P(O)(OH)₂, groups of formula —O—P(O)(OH)(OR), and groups of formula —O—P(O)(OR)₂, wherein R may be chosen from H, inorganic cations, and organic cations. Non-limiting examples of inorganic cations include alkali metals, such as, for example, potassium lithium, and sodium. A non-limiting example of organic cations is at least one additional silicone compound which may be identical to or different from the at least one silicone compound bonded to the other oxygen of the phosphate group.

In one embodiment, the at least one silicone compound comprising at least one phosphate group is chosen from silicone compounds of formula (I): 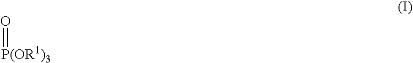
wherein R¹, which may be identical or different, are each chosen from H, organic cations, inorganic cations, optionally substituted hydrocarbons (such as alkyl groups and alkenyl groups comprising from 1 to 22 carbon atoms), optionally substituted aromatic groups; groups of formula (II) and salts thereof:
CH₃(CH₂)ₓ—O-(EO)_{c}—(PO)_{d}-(EO)ₑ—CH₂CH₂— (II)
wherein: c, and d, which may be identical or different, are each integers ranging from 0 to 20; e is an integer ranging from 0 to 19; and x is an integer ranging from 0 to 21; groups of formula (III) and salts thereof:
HO-(EO)_{c}—(PO)_{d}-(EO)ₑ—(CH₂)ₓ— (III)
wherein: c, d, and e, which may be identical or different, are each integers ranging from 0 to 20; and x is an integer ranging from 0 to 21; and groups of formula (IV) and salts thereof: 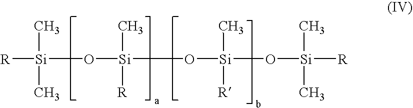
wherein: a is an integer ranging from 0 to 200; b is an integer ranging from 0 to 200; R′, which may be identical or different, are each chosen from optionally substituted hydrocarbons, such as alkyl groups and alkenyl groups comprising from 1 to 22 carbon atoms, optionally substituted aromatic groups, groups of formula (III) as defined above and salts thereof; and R, which may be identical or different, are each chosen from optionally substituted hydrocarbons, such as alkyl groups and alkenyl groups comprising from 1 to 22 carbon atoms, optionally substituted aromatic groups, optionally substituted divalent hydrocarbons, such as alkylene groups and alkenylene groups comprising from 1 to 22 carbon atoms, optionally substituted divalent aromatic groups, groups of formula (III) as defined above and salts thereof, and groups of formula (V):
-(EO)_{c}—(PO)_{d}-(EO)ₑ—(CH₂)₃— (V)
wherein: the (CH₂) ₃ end is bonded to the silicon of the compound of formula (IV) and the (EO) or (PO) end, if present, is bonded to the oxygen of the compound of formula (I); c, d, and e, which may be identical or different, are each integers ranging from 0 to 20; EO is an ethylene oxide group; and PO is a propylene oxide group; and with the proviso that at least one R is chosen from groups of formula (V) and salts thereof; and with the further proviso that at least one R¹ is chosen from groups of formula (IV) and salts thereof and at least one other R¹ is chosen from H, organic cations, and inorganic cations.

Non-limiting examples of the inorganic cations include alkali metals, such as potassium, lithium, and sodium. Non-limiting examples of the at least one silicone compound include those commercially available from Phoenix Chemical, Inc. of New Jersey under the name of Pecosil®, such as Pecosil® PS-100, Pecosil® PS-112, Pecosil® PS-150, Pecosil® PS-200, Pecosil® WDS-100, Pecosil® WDS-200, Pecosil® PS-100 B, and Pecosil® PS-100 K and those commercially available from Siltech under the name Silphos A-100 and Silphos A-150. Other non-limiting examples of the at least one silicone compound include those described in U.S. Pat. Nos. 5,070,171, 5,093,452, and 5,149,765 the disclosures of which are incorporated herein by reference.

Suitable silicone sulfates for use in the present invention include those represented by formula VI: 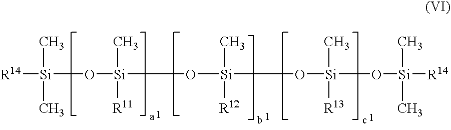
wherein R¹¹ is selected from lower alkyl having one to eight carbon atoms or phenyl, R¹² is —(CH₂)₃—O-(EO)ₓ—(PO)_{y}--(EO)_{z}—SO₃³¹-M⁺ wherein M is a cation and is selected from Na, K, Li, or NH₄; x, y and z are integers independently ranging from 0 to 100; R¹³ is —(CH₂)₃—O-(EO)ₓ—(PO)_{y}-(EO)_{z}—H; R¹⁴ is methyl or hydroxyl; a¹ and c¹ are independently integers ranging from 0 to 50; b¹ is an integer ranging from 1 to 50. An example thereof is Ultrasil SA-1 silicone commercially available from Noveon.

Suitable silicone sulfosuccinates which may be employed include, but are not limited to, those corresponding to formula VII: 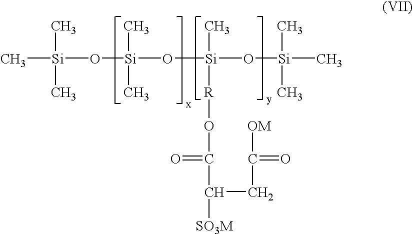
wherein R represents a divalent radical selected from 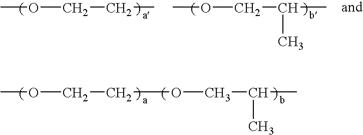
wherein a′ and b′ range from 0 to 30; x and y are such that the molecular weight ranges from 700 to 1600, and M is an alkali metal such as sodium or potassium, or an ammonium group.

A particularly preferred anionic silicone is Dimethicone PEG-8 phosphate, commercially available from Noveon under the tradename Ultrasil PE-100.

The anionic silicone is present in the composition in an amount ranging from greater than 0 to 50% by weight, preferably from greater than 0 to 30% by weight, and more preferably from greater than 0 to 15% by weight, based on the weight of the composition as a whole.

It has surprisingly been found that the carrier composition of the present invention facilitates the formulation of an aqueous delivery system capable of carrying up to 50% by weight, preferably up to 30% by weight, more preferably up to 20% by weight, and most preferably up to 10% by weight, all weights being based on the weight of the composition, of water-insoluble ingredients. The resultant aqueous delivery system is both stable, and clear to slightly hazy/limpid in appearance.

Thus, in another aspect, the present invention relates to an aqueous delivery system comprising the carrier composition, at least one water-insoluble ingredient and an aqueous phase. The carrier composition is present in an amount sufficient to allow the at least one water-insoluble material to be incorporated into the aqueous system. The amount sufficient for incorporation may vary depending on the type of composition; for example, shampoo and mascara formulations require a lower concentration of the carrier composition than do conditioner, deep treatment, bleach, permanent wave, dye, and relaxant compositions.

Water-insoluble materials or ingredients include, but are not limited to, the following:

(1) Lipophilic “ingredients” or “materials” such as silicones, oil-soluble vitamins such as Vitamin E and Vitamin A, sunscreens, ceramides and natural oils: The lipophilic ingredients may be in the form of sunscreens, bacteriostats, moisturizers, colors, topical pharmaceuticals and the like. Preferred lipophilic ingredients include: Vitamin E, Vitamin E Acetate, Vitamin A Palmitate, olive oil, mineral oil, 2-oleamido-1,3-octadecanediol, octylmethoxy cinnamate, octyl salicylate, and silicones such as dimethicone, cyclomethicone, phenyl trimethicone, dimethiconol, dimethicone copolyol, aminosilicone and laurylmethicone copolyol. The lipophilic ingredients will, for example, moisturize or condition the skin, hair, and/or eyelashes and leave behind no oily feel.

(2) Water-insoluble polymers, resins, and latexes, wherein the polymers and resins include but are not limited to those containing carboxyl moieties, such as acrylates and other carboxy polymers.

Preferred water-insoluble ingredients for use in the present invention include silicones ranging from low molecular weight fluids to high molecular weight gums; hydrocarbons such as mineral oil, petrolatum, paraffins, iso-paraffins, aromatic hydrocarbons, and the like; plant oils such as olive, avocado, coconut, and the like; fatty acids; fatty esters; fatty alcohols; and fatty waxes.

The aqueous phase of the inventive delivery system can contain additional ingredients such as anionic surfactants, organic salts, inorganic salts, proteins, hair dyes, water-soluble polymers, quaternary ammonium compounds, complex and simple carbohydrates, amino acids, preservatives and fragrances.

Another embodiment of the present invention is drawn to a process for making an aqueous delivery system. This process involves: (a) providing a carrier composition containing at least one phospholipid, at least one nonionic surfactant, and at least one anionic silicone; (b) providing at least one water-insoluble ingredient; (c) optionally, heating the carrier composition to form a heated composition; (d) adding the water-insoluble ingredient to either the carrier composition, the heated composition or both; (e) providing an aqueous solution; (f) adding the aqueous solution to the heated mixture to form a diluted mixture and (g) cooling the diluted mixture to form the desired aqueous delivery system. Preferably the aqueous delivery system obtained can carry a high load (i.e., 50% is considered a high load) of the water-insoluble ingredient.

In another embodiment, the present invention is drawn to a process for treating keratinous substances such as, but not limited to, hair, skin, or eyelashes by contacting the keratinous substance with the above-disclosed aqueous delivery system. The term treating in the context of this invention includes, but is not limited to, shampooing, conditioning, dyeing, bleaching, permanent waving, relaxing, setting, moisturizing, and making-up, for example, applying mascara or foundation.

As mentioned previously, the carrier composition and aqueous delivery system of the present invention can be used as an ingredient itself in, for example, shampoos, conditioners (rinse-off and leave-in), deep treatments for hair, body washes, bath gels, hair dyeing compositions, permanent wave formulations, relaxers, make-up preparations, particularly mascara and foundation, and skin creams or lotions.

The aqueous delivery systems of the invention can be further associated, in the hair products described above, with proteins including hydrolyzed soy protein, lauryldimonium hydrolyzed soy protein (cationic Soya protein) and wheat amino acids. The proteins could also include corn, wheat, milk, or silk proteins, collagens, keratins, or others. Furthermore, taurine and arginine hydrochloride may be associated therein to maximize protein binding to the keratinous substrate. Cationic proteins or proteins in general may be stabilizers for the aqueous delivery system and enhance its delivery by changing the charge of the aqueous delivery system. The skin and the hair attract cationic ingredients, and proteins are generally substantive to these tissues.

Other ingredients in the aqueous delivery system may include cationic polymers, such as polyquaternium 4, polyquaternium 6, polyquaternium 7, polyquaternium 10, polyquaternium 11, polyquaternium 16, polyquaternium 22, and polyquaternium 32, cationic conditioners, such as quaternium 27, behenamidopropyl PG-dimonium chloride, hydroxyethyl tallowdimonium chloride, hexadimethrine chloride, stearalkonium chloride, and cetrimonium chloride, isoparaffins, sodium chloride, propylene glycol, preservatives such as phenoxyethanol, methylparaben, ethylparaben, and propylparaben, pH adjusters such as phosphoric acid, humectants such as trehalose, and emollients such as octyldodecanol. Many other examples of materials from the classes listed above would be readily known to one of ordinary skill in the art.

Further, shampoos, conditioners, and deep treatments within the scope of the present invention may be used on hair which has been treated, e.g., with color (dye or bleach) or chemicals (permanent wave or straightening), or which is dry or fine and show significant substantivity for the hair.

The invention will be further clarified by the following examples, which are intended to be illustrative of the invention, but not limiting thereof.

### EXAMPLES

General procedure: The general procedure for formulating carrier system is as follows: First, add deionized water to the beaker. Begin mixing at high speed. Next, slowly disperse lecithin. Mix well until lecithin is free of clumps. Then reduce mixing speed to moderate speed, and begin heating the batch to 80° C. At 75° C., add Procetyl AWS (INCI: PPG-5 Ceteth-10) and Ultrasil PE-100 (silicone phosphate). Mix well. At 80° C., add active ingredient. Mix well until formula is clear, maintaining at 80° C. Cool to room temperature. If necessary, pour at 75° C. since formula may gel up around 70° C.

### Example 1

### The Carrier System

Following the general procedure, a series of solutions were made as depicted in Table 1. The results show that a complete carrier system is needed to form a clear, stable solution that contains a water insoluble silicone.

TABLE 1
Experiments showing a clear, stable carrier system

| Combinations of ingredients | Appearance |
|---|---|
| Water 46 g + Lecithin 5 g + DC 200 (300,000 cst) | Hazy |
| 4 g | |
| Water 46 g + Procetyl AWS 25 g + DC 200 | Hazy |
| (300,000 cst) 4 g | |
| Water 46 g + Silicone Phosphate 20 g + DC 200 | Hazy |
| (300,000 cst) 4 g | |
| Water 46 g + Lecithin 5 g + Procetyl AWS | Hazy |
| 25 g + DC 200 (300,000 cst) 4 g | |
| Water 46 g + Lecithin 5 g + Silicone Phosphate | Hazy |
| 20 g + DC 200 (300,000 cst) 4 g | |
| Water 46 g + Procetyl AWS 25 g + Silicone | Hazy |
| Phosphate 20 g + DC 200 (300,000 cst) 4 g | |
| Water 46 g + Lecithin 5 g + Procetyl AWS 25 g + | Clear |
| Silicone Phosphate 20 g + DC 200 (300,000 cst) 4 g | |

### Example 2

### The Carrier System with Different Silicones

The carrier system is able to carry different types of silicones, such as DC 200 (INCI: dimethicone), DC 556 (INCI: phenyltrimethicone), and DC 5562 (INCI: Bis-hydroxyethoxypropyl dimethicone). When formulated using the general procedure as described above, clear, stable systems are obtained, as shown in table 2.

TABLE 2
Carrier system with different types of silicones

| | A | B | C | D | E |
|---|---|---|---|---|---|
| D.I. Water | 46% | 46% | 46% | 46% | 46% |
| Lecithin | 5% | 5% | 5% | 5% | 5% |
| PPG-5 Ceteth-20 | 25% | 25% | 25% | 25% | 25% |
| Silicone phosphate | 20% | 20% | 20% | 20% | 20% |
| Silicones | 4% | 4% | 4% | 4% | 4% |
| | (DC 200 | (DC 200 | (DC 200 | (DC 556) | (DC 5562 |
| | (1000 cst)) | (60,000 cst)) | (300,000 cst)) | | Carbinol fluid) |

Furthermore, the above formula D is dilutable in water and in a shampoo base (25% TEA Lauryl sulfate, 10% Cocamidopropyl Betaine, and 65% DI Water).

### Example 3

### Carrier System with Different Oils

The carrier system is able to carry different types of oils, such as mineral oil and tea tree oil. When formulated using the general procedure as described in above, clear, stable systems are obtained as shown in table 3.

TABLE 3
Carrier system with different types of oils

| | A | B |
|---|---|---|
| D.I. Water | 46% | 46% |
| Lecithin | 5% | 5% |
| PPG-5 Ceteth-20 | 25% | 25% |
| Silicone Phosphate | 20% | 20% |
| Oils | 4% | 4% |
| | (Mineral oil) | (Tea tree oil) |

Furthermore, the above formulas A and B are dilutable in water and in a shampoo base (25% TEA Lauryl sulfate, 10% Cocamidopropyl Betaine, and 65% DI Water).

### Example 4

### Carrier System with Esters/Waxes/Hydrocarbons

The carrier system is able to carry different types of esters, such as capric/caprylic triglyceride, different types of waxes, such as Phytowax olive 6L25 (INCI: hydrogenated hexyl olive esters), and different types of hydrocarbons, such as C11-13 Isoparaffin. When formulated using the general procedure as described above, clear, stable systems are obtained, as shown in table 4.

TABLE 4
Carrier system with different types of esters/waxes/hydrocarbons

| | A | B | C |
|---|---|---|---|
| D.I. Water | 46% | 46% | 46% |
| Lecithin | 5% | 5% | 5% |
| PPG-5 Ceteth-20 | 25% | 25% | 25% |
| Silicone Phosphate | 20% | 20% | 20% |
| Ester/wax/ | 4% | 4% | 4% |
| hydrocarbon | (Capric/caprylic | (Phytowax | (C11-13 |
| | triglyceride) | olive 6L25) | Isoparaffin) |

Furthermore, all of the above formulas are dilutable in a shampoo base (25% TEA Lauryl sulfate, 10% Cocamidopropyl Betaine, and 65% DI Water). In addition, the above formulas A and B are dilutable in water.

### Example 5

### Carrier with other Phospholipid

Carrier system is also clear and stable when Lecithin is replaced with another biomemetic phospholipid, such as Phospholipid EFA (INCI: Linoleamidopropyl PG-dimonium chloride phosphate) (30% active) when formulated using C11-13 Isoparaffin. See table 5.

TABLE 5
Carrier system with Phospholipid EFA
carrying C11-13 Isoparaffin

| | |
|---|---|
| D.I. Water | 46% |
| Phospholipid EFA | 5% |
| PPG-5 Ceteth-20 | 25% |
| Silicone Phosphate | 20% |
| C11-13 Isoparaffin | 4% |

Furthermore, the above formula is dilutable in a shampoo base.

### Example 6

### Measure of Conditioning Effect Using the Carrier System

The conditioning effect of an aqueous carrier system containing the carrier composition of the present invention, as a carrier of Mineral Oil, was assessed by the wet combability method using the Instron 4444 Tensile Tester. Evaluation was carried out on bleached hair, cleansed with 15% SLES, and treated a single time with the aqueous delivery system. Specifically, swatches were treated once with 0.3g product per gram of hair. The hair was treated with the product for 1 minute and rinsed for 10 seconds. The total combing energy required to comb the hair was measured after cleansing with SLES (Wᵢ), as well as after treatment (W_{f}). The percent change in combing energy was calculated using the following formula:
% Δ Combing Energy=(W_{f}−Wᵢ)/(Wᵢ)×100%
where Wᵢ=combing energy required prior to treatment and W_{f}=combing energy required after treatment.

Treatments, which improve wet combability, will result in negative percent change values.

A clear, stable aqueous delivery system composed of the following formula was used to treat the hair:

| Classification | Trade Name | % wt/wt |
|---|---|---|
| Water | Water | 46 |
| Silicone Phosphate | Ultrasil PE-100 | 20 |
| Lecithin | Emulmetik 100 | 5 |
| Nonionic Surfactant | Procetyl AWS | 25 |
| Mineral Oil | Carnation White Mineral Oil | 4 |
| | | 100 |

Following a single application of the aqueous delivery system, the percent change in combing energy was −22.24%, indicating that the treated hair is significantly more conditioned. Thus, treatment with the aqueous delivery system made the hair more manageable and easier to comb by 22.24%.

It will be apparent to those skilled in the art that various modifications and variations can be made in the delivery system, composition and methods of the invention without departing from the spirit or scope of the invention. Thus, it is intended that the present invention cover the modifications and variations of this invention provided that they come within the scope of the appended claims and their equivalents.

What is claimed is:

1. A composition comprising:
(a) at least one phospholipid;
(b) at least one nonionic surfactant;
(c) at least one anionic silicone; and
(d) at least one water-insoluble material.

2. The composition of claim 1 wherein (a) is lecithin.

3. The composition of claim 1 wherein (a) is present in an amount of from about greater than 0 to about 30% by weight, based on the weight of the composition.

4. The composition of claim 1 wherein (a) is present in an amount of from about greater than 0 to about 5% by weight, based on the weight of the composition.

5. The composition of claim 1 wherein (b) has an HLB of at least about 8.

6. The composition of claim 1 wherein (b) is present in an amount of from about greater than 0 to about 70% by weight, based on the weight of the composition.

7. The composition of claim 1 wherein (b) is present in an amount of from about greater than 0 to about 20% by weight, based on the weight of the composition.

8. The composition of claim 1 wherein (c) is a silicone phosphate.

9. The composition of claim 1 wherein (c) is a silicone carboxylate.

10. The composition of claim 1 wherein (c) is-a silicone sulfate.

11. The composition of claim 1 wherein (c) is present in an amount of from about greater than 0 to about 50% by weight, based on the weight of the composition.

12. The composition of claim 1 wherein (c) is present in an amount of from about greater than 0 to about 15% by weight, based on the weight of the composition.

13. The composition of claim 1 wherein (d) is present in an amount of up to about 50% by weight, based on the weight of the composition.

14. The composition of claim 1 wherein (d) is present in an amount of up to about 10% by weight, based on the weight of the composition.

15. A process for making an aqueous delivery system which is both stable, and clear to slightly limpid in appearance, involving the steps of:
(a) providing a carrier composition containing: (i) at least one phospholipid, (ii) at least one nonionic surfactant and, (iii) at least one anionic silicone;
(b) providing at least one water-insoluble ingredient;
(c) optionally, heating the composition of step (a) to form a heated mixture;
(d) adding (b) to either step (a), step (c) or both step (a) and (c);
(e) adding an aqueous solution to the heated mixture to form a diluted mixture; and
(f) cooling the diluted mixture to form the aqueous delivery system.

16. A process for treating a keratinous substrate comprising contacting the keratinous substrate with a composition containing:
(a) at least one phospholipid;
(b) at least one nonionic surfactant;
(c) at least one anionic silicone; and
(d) at least one water-insoluble material.

17. The process of claim 16 wherein (a) is lecithin.

18. The process of claim 16 wherein (a) is present in an amount of from about greater than 0 to about 30% by weight, based on the weight of the composition.

19. The process of claim 16 wherein (a) is present in an amount of from about greater than 0 to about 5% by weight, based on the weight of the composition.

20. The process of claim 16 wherein (b) has an HLB of at least about 8.

21. The process of claim 16 wherein (b) is present in an amount of from about greater than 0 to about 70% by weight, based on the weight of the composition.

22. The process of claim 16 wherein (b) is present in an amount of from about greater than 0 to about 20% by weight, based on the weight of the composition.

23. The process of claim 16 wherein (c) is a silicone phosphate.

24. The process of claim 16 wherein (c) is a silicone carboxylate.

25. The process of claim 16 wherein (c) is a silicone sulfate.

26. The process of claim 16 wherein (c) is present in an amount of from about greater than 0 to about 50% by weight, based on the weight of the composition.

27. The process of claim 16 wherein (c) is present in an amount of from about greater than 0 to about 15% by weight, based on the weight of the composition.

28. The process of claim 16 wherein (d) is present in an amount of up to about 50% by weight, based on the weight of the composition.

29. The process of claim 16 wherein (d) is present in an amount of up to about 10% by weight, based on the weight of the composition.

30. The process of claim 16 wherein the keratinous substrate is hair.

31. A personal care composition comprising the composition of claim 1.

32. The composition of claim 31 wherein the composition is chosen from a shampoo, a conditioner, a deep treatment, or a combination shampoo and conditioner.
